(19) United States
(12) Patent Application Publication
(10) Pub. No.: US 2019/0206570 A1
(43) Pub. Date: Jul. 4, 2019

(54) Remote View Playback Tool

(71) Applicant: Talis Clinical LLC, Streetsboro, OH (US)

(72) Inventors: Gary Colister, Hudson, OH (US); Bishoy Magdalla, Hudson, OH (US); Giuseppe Saracino, Gainsville, FL (US); William Murphy, Aurora, OH (US); Harish Lecamwasam, Scottsdale, AZ (US)

(21) Appl. No.: 16/239,077

(22) Filed: Jan. 3, 2019

Related U.S. Application Data
(60) Provisional application No. 62/613,218, filed on Jan. 3, 2018.

(51) Int. Cl. G16H 50/20; G06F 16/22; G06F 16/215; G16H 10/60; G16H 40/67; G16H 80/00

(52) U.S. Cl. CPC G16H 50/20; G06F 16/22; G16H 80/00; G16H 10/60; G16H 40/67; G06F 16/215

(57) ABSTRACT

An apparatus and method for remotely monitoring and reviewing records of medical patients to gain actionable insights including a data collection, storage and access system and a telecommunications network, at least one monitoring station for monitoring data elements and for sending the monitored data elements to a command center via the telecommunications network, a patient database, a data storage engine for collecting data from any number of electronic devices and instructions for transmitting data, as well as instructions for storing data associated with the patient records, at least one communication hub, where the telecommunication network providing access to all data, including continuous waveform data, collected during the treatment of the patient including quality data not included in any patient record storage location, and a user interface rules engine that provides the user with ability to select any point in time during the patient treatment to facilitate review the details collected regarding the treatment that the selected time, review the details, creating guidance rules to identify cases identified as complying with the defined rules, to define who, when and how to communicate that cases meeting the defined criteria are available for review, in this case the who may be only the individual(s) evaluating the defined criteria without notification to anyone monitoring a current case, or including specific individuals monitoring the current case.

### BACKGROUND OF THE INVENTION

The present invention is directed to a remote view playback tool, which facilitates, for example, converting patient vital signs into waveforms to facilitate remote monitoring of patients and improve decisions regarding patient care.

Medical devices actively monitoring patient vital signs often display waveforms. The waveforms and corresponding numeric values are used to make decisions regarding the patient care at the point of care. However, today there are limited means to collect and store these waves forms in patient records. When saved the saved information appears as a screen shot. This significantly limits the effectiveness of telemedicine interaction with remote specialists and its use during after the fact assessments of never events.

To fully support telemedicine and after the fact assessments of specific patient care procedures, there is a real need to be able to have access to the entire waveform data collected during the patient care. This supports the assessment of the patient responses to various potential contributors during the patient care. Without the ability to select specific times during the patient care to assess the entire history of the patient care, the assessments are limited to an incomplete picture of the whole story.

Today telemedicine is used to support remote ICU bunkers. The intensivist monitoring these ICU patients have access to a video feed that shows the actual medical device active monitoring screens. This approach provides only the current vital signs. There is no ability to review earlier details to fully assess the potential contributions to the current patient condition. Remote view provides a means to store vital signs including waveforms in the patient record. These records may be less than a second behind the active patient monitor screens. Once stored the remote view allows the remote practitioner to easily move back and forth through the records to fully assess the needs of the patient and when appropriate provide guidance to the practitioner.

Today there is no easy-way for a physician to have access to the most current condition of their patients remotely. Remote view provides the physician or other authorized practitioners to readily see the patient condition in near real time, as well as the ability to review the patient's condition leading up to the current status. The alternative today is attempting to contact someone on duty and have them review the patient's condition over the phone, creating opportunities for miscommunications. Remote view supports telemedicine by providing the remote practitioner with near real time access the patient's record and ability to review all vital signs, patient care activities and patient response to the treatment leading up to the current condition. This provides for a more complete understanding of the patient condition. An example of this is as Anesthesiologist supervising multiple CRNA's. Remote view provides the supervisor to review the facts regarding a specific patient while walking to the OR to assist the CRNA. Thus, reducing the time required to review the patient status with the CRNA prior to taking the appropriate actions to address the situation.

Access to waveform data would be helpful where Medical Facilities have an obligation to focus on improving patient outcomes. Additionally, when Serious or Never Events occur there is requirement to review the specific conditions leading up to the event. Where possible take actions to prevent recurrence. The failure investigation process is focused identifying the root cause(s) of the event. It is not uncommon to identify several potential root cause(s). Effective failure investigation requires the ability to assess the impact of all patient interactions and the patient response to those interactions. However, even electronic patient records are limited to a series of snap shots in time during patient care. The snap shots routinely do not adequately provide a means to assess the patient response to the interactions. Therefore, the determination of correlation between any specific patient interaction is often based on opinions based on prior experience, but not based on scientifically supported facts.

The limitation placed on the failure investigation process is the content of the patient care record. This is especially true with paper records and/or electronic medical records that focus on procedural and billing documentation. The patient records limitations include the information included in the patient record does not provide enough details to clearly assess the relevant information available to the practitioner at the time of the event or prior to the event. The limitations could include, for example, the numeric values of patient vital signs, the waveform information, the specific time lines for interactions, and access to data not contained in the patient record

Numeric values of patient vital signs displayed on medical device screens are not collected frequently enough to properly assess the patient response to a specific interaction with the patient or advanced knowledge of patient condition. Waveform information displayed on medical devices may not be part of the patient record. Even when present, the content is a series of snap shots at prescribed intervals. The snap shots do not routinely provide the details needed. Specific time lines show all patient interactions with patient vital signs before and after each patient interaction. Any ability to access any past data is limited if it not contained in the patient record. Because, for the most part, the current systems do not collect the data, it is not available for subsequent review.

Once the root cause(s) have been identified the process requires the identification of potential corrective/preventive actions that will eliminate or at least substantially eliminate the root cause(s). To accomplish this the potential-actions need to be assessed and prior to implementation verify and validate that the actions are effective in eliminating/substantially reducing the root causes, without creating new potential issues. However, there is no means to assess with validity of these proposed corrective actions without exposing some number of patients to potential increase of risk. Running these experiments would require at a minimum a formal Internal Review Board (IRB) review and approval. In many situations, the proposals may not align with the approved procedural protocols. Therefore, the IRB may question the wisdom of interfering with accepted practice without scientific evidence. The IRB will deny the approval of the experiment because the proposed changes cannot the adequately justified. It should be noted that not all protocol changes require IRB review. However, best practice in commonly accepted process control tools would require some form of justification to access the effectiveness of the proposed changes.

The tools currently available are limited with respect their ability to support traditional failure investigation, process control methodology and verification and validation of proposed corrective and preventive actions. For example, U.S. Pat. No. 7,315,825 to Rosenfeld et al teaches a rules-base patient care system for use in remote monitoring healthcare locations with the purpose of supporting telemedicine during patient treatment. A patient rules generator creates rules for the patients. The rules generator acquires performance measures indicative of the ability of a rule to predict changes in the condition of the patient. A determination is made from the rules performance measures whether to revise the rule. A rules engine applies a rule to selected data elements stored in the database to produce an output indicative of a change in the medical condition of the patient. The output from the rules engine is used to determine if intervention is warranted. But, the remote monitoring only involves a current review. It does not allow for a retrospective view. Additionally, these rules engines may not be understood by the practitioner and may not include all the data available from multiple medical devices that add critical insight into the proper care of the patient.

Medical devices monitoring patient vital signs often display waveforms. The waveforms and corresponding numeric values are used to make decisions regarding the patient care. However, today there are limited means to collect and store these waves forms in patient records. When saved the saved information appears as a screen shot. This significantly limits the effectiveness of telemedicine interaction with remote specialists and its use during after the fact assessments of never events.

Medical devices monitoring patient vital signs often display waveforms. The waveforms and corresponding numeric values can be critical to make decisions regarding the patient care. However, today there are no or very limited means to collect and store these waves forms in patient records. When saved the saved information appears as a screen shot. This significantly limits the effectiveness of telemedicine interaction with remote specialists and its use during after the fact assessments of never events. While near real time video is reviewed remotely, there is no ability to review the recent history previously shown on the active patient monitor, or review all data provided by the medical device during the case.

To fully support telemedicine and after the fact assessments of specific patient care procedures, there is a real need to be able to have access to the entire collected during the patient care, including the waveform data. This supports the assessment of the patient responses to various potential contributors during the patient care. Without the ability to select specific times during the patient care to assess the entire history of the patient care, the assessments are limited to an incomplete picture of the whole story.

### SUMMARY OF THE INVENTION

The present invention is directed to a remote view playback tool or system and method for medical patients to improve patient outcomes which includes a data storage and access system and a telecommunications network; at least one monitoring station comprising monitoring equipment where the monitoring equipment includes instructions for monitoring data elements and for sending the monitored data elements via the telecommunications network, and includes instructions for receiving monitored data elements from patients and accessing patient data elements indicative of a medical conditions associated with each of the patients; a patient database containing information concerning the medical condition, history, and status of each of the patients; at least one communication hub comprising instructions for collecting data from any number of electronic devices including medical devices and instructions for transmitting data to any number of electronic devices including medical devices, as well as instructions for storing data associated with the patient records and/or data to be stored as quality data; a data storage engine comprising a means for collecting data from any number of electronic devices including medical devices and instructions for transmitting data to any number of electronic devices including medical devices, as well as instructions for storing data associated with the patient records and/or data to be stored as quality data; the telecommunication network providing access to all data, including continuous waveform data, collected during the treatment of the patient and quality data not included in any patient record storage location; and a user interface rules engine that provides the user with ability to select any point in time during the patient treatment to review the details collected regarding the treatment at the selected time, review the details before or after the selected time, create guidance rules to identify cases identified as complying with the defined rules, the user may define the period of time used to identify the cases for review, the user may select future cases only, or past cases to some defined date, or a combination of the two, and define who, when and how to communicate that cases meeting the defined criteria are available for review, in this case the who may be only the individual(s) evaluating the defined criteria without notification to anyone monitoring a current case, or including specific individuals monitoring the current case; where the user interface rules engine includes means to collect, store and process data in near real-time, means to compose views that organize data for end-users to consume, means to let a user create execution steps on the data streams, means to notify end-users based on execution steps defined by end-users, means to display an organized view of data within a timeline of events, means for end-users to change or augment the execution steps, and means to provide notifications at the same time the end-user is reviewing data, so that data from multiple disparate sources can be acquired and consolidated within a unified view, and the data can be reviewed remotely to make decisions about patient care.

The tool of the present invention allows the remote access to near real time patient records, including data not necessarily included in the EMR. The remote access may include data available to the attending practitioner from the medical device active patient monitors in addition to the EMR data which is a subset of the complete data set. The tool may be used to create guidance rules to identify cases identified as complying with the defined rules, to define who, when and how to communicate that cases meeting the defined criteria are available for review, in this case the who may be only the individual(s) evaluating the defined criteria without notification to anyone monitoring a current case, or including specific individuals monitoring the current case.

### DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is a flow diagram of a global view of the continuous improvement system of the present invention;

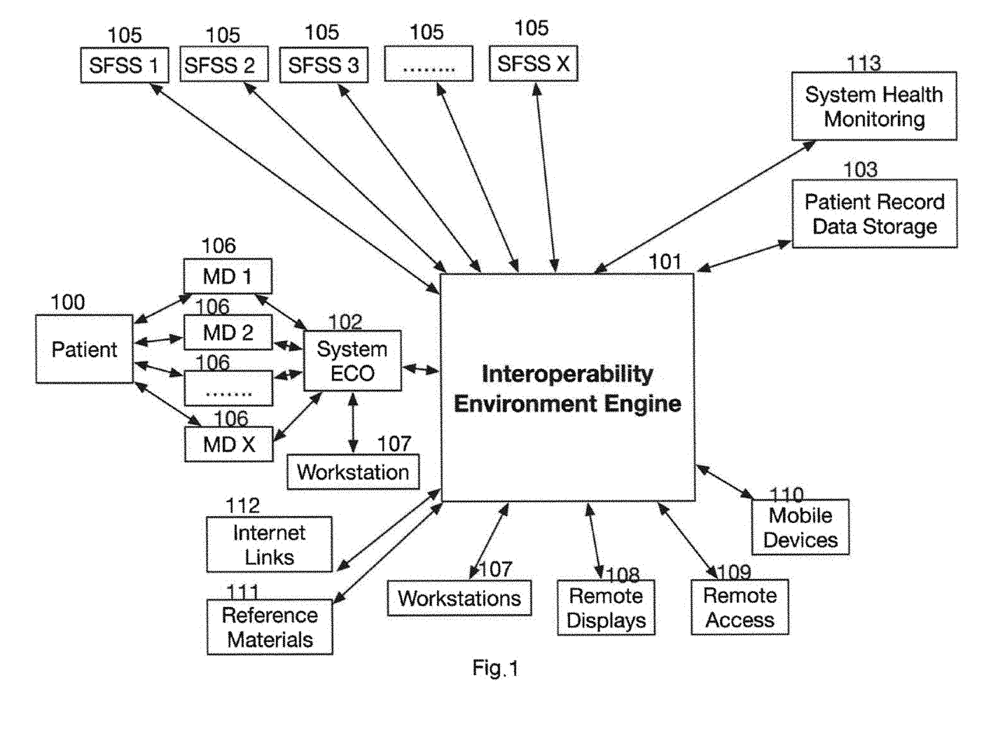

FIG. 2 illustrates the flow of a decision support algorithm in accordance with the present invention;

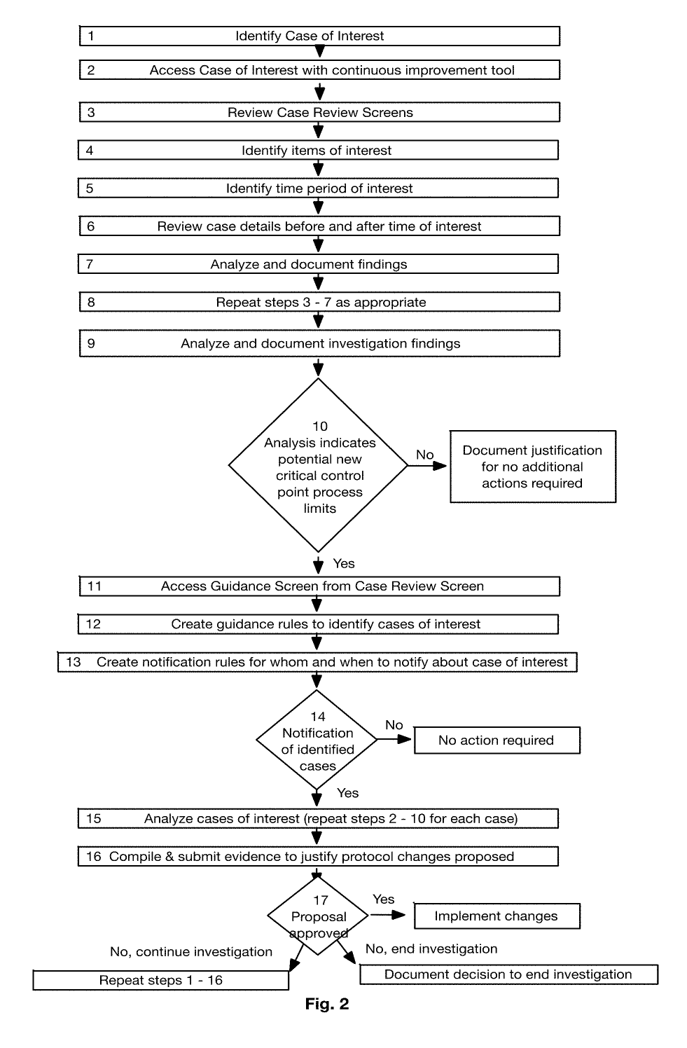

FIG. 3 is a screen shot showing a case analysis review screen;

FIG. 4 is a screen shot showing a case analysis review screen showing interaction with data options;

FIG. 5 is a screen shot showing a case analysis review screen which includes waveform displays;

FIG. 6 is a screen shot showing a case analysis review screen showing a guidance tool to create or modify a guidance;

FIG. 7 is a screen shot showing a case analysis review screen showing the selection of a primary filter;

FIG. 8 is a screen shot showing a case analysis review screen indicating that the event is an incision;

FIG. 9 is a screen shot showing a case analysis review screen which provides an airway summary;

FIG. 10 is a screen shot showing a case analysis review screen showing that the anesthesia is general, as well as the secondary and third level filters; and

FIG. 11 is a screen shot showing a case analysis review screen which defines the scope of the fourth level filter.

### DETAILED DESCRIPTION OF THE INVENTION

The present invention is directed to a remote view playback tool, to facilitate remote monitoring and review of patient's medical data and trends and improve decisions regarding patient care. For example, the present invention facilitates converting patient vital signs into waveforms which can be stored with patent records and may be displayed and played for review.

The present invention identifies a need to be able to review specific interactions with patient and patient response by being able to move back and forward in the patient record. To fully accomplish this there is a need to be able to replay the waveforms collected. The present invention is to a simple means to easily view remotely the historic waveform. Historic here is defined as any delay from the active patient monitoring. Therefore, using commonly accepted telemedicine practices, the active practitioners may consult with remotes specialists if needed.

The remote screens support the ability to move back in time during the current patient care under review. The same screens may also be utilized to evaluate the case during formal reviews. These screens also include icons to show when specific patient interactions were performed.

The waveform data is collected directly from the medical devices. These waveforms are stored with the patient records. Once stored the waveforms may be displayed and played on a remote device. With the ability to play the stored waveforms the system is also includes to play, replay, fast forward or fast reverse to identify the time periods of most interest to the medical practitioner reviewing these records.

The present invention includes a telecommunications network; at least one monitoring station comprising monitoring equipment where the monitoring equipment includes instructions for monitoring data elements and for sending the monitored data elements via the telecommunications network, and includes instructions for receiving monitored data elements from patients and accessing patient data elements indicative of a medical conditions associated with each of the patients; a patient database containing information concerning the medical condition, history, and status of each of the patients; at least one communication hub comprising instructions for collecting data from any number of electronic devices including medical devices and instructions for transmitting data to any number of electronic devices including medical devices, as well as instructions for storing data associated with the patient records and/or data to be stored as quality data; a data storage engine comprising a means for collecting data from any number of electronic devices including medical devices and instructions for transmitting data to any number of electronic devices including medical devices, as well as instructions for storing data associated with the patient records and/or data to be stored as quality data; the telecommunication network providing access to all data, including continuous waveform data, collected during the treatment of the patient and quality data not included in any patient record storage location; and a user interface rules engine that provides the user with ability to select any point in time during the patient treatment to review the details collected regarding the treatment at the selected time, review the details before or after the selected time, create guidance rules to identify cases identified as complying with the defined rules, the user may define the period of time used to identify the cases for review, the user may select future cases only, or past cases to some defined date, or a combination of the two, and define who, when and how to communicate that cases meeting the defined criteria are available for review, in this case the who may be only the individual(s) evaluating the defined criteria without notification to anyone monitoring a current case, or including specific individuals monitoring the current case; where the user interface rules engine includes means to collect, store and process data in near real-time, means to compose views that organize data for end-users to consume, means to let a user create execution steps on the data streams, means to notify end-users based on execution steps defined by end-users, means to display an organized view of data within a timeline of events, means for end-users to change or augment the execution steps, and means to provide notifications at the same time the end-user is reviewing data, so that data from multiple disparate sources can be acquired and consolidated within a unified view, and the data can be reviewed remotely to make decisions about patient care.

The present invention allows for the acquisition of data from multiple disparate sources, consolidation of all information within a unified view, running process controls/workflows and delivering actionable insights to specific users in near-real time. The present system identifies which information is actionable based on user criteria, supplied by the user, and a decision support algorithm provided by management software. Actionable information can be presented with rich context compared to typical isolated ancillary systems. The information can be personalized to patient, clinical and/or admin user. Users can replay the timeline of events with all relevant information under a given context. Information can be added or deleted to sharpen context, to gain additional knowledge, or to immediately implement changes based on the review.

The novel process improvement tool enables collection, storage and automation of process controls and/or workflows on vast disparate data streams that normally are not accessible with paper records or poorly accessible due to isolated ancillary data systems with current electronic systems. Workflow automation changes and data points collected can be easily augmented. The improvement tool of the present invention gives users unique ability to dynamically organize data into views of patient populations regardless of geographic location, with additional ability to pare view according to user specified criteria and the ability to collect data from devices, HIT systems, external sources, user input, or any other source of data which can be made available in electronic format.

The data entry can be a mix of automated and user input. The present invention has the ability to use any of the above to create a set of evaluations on the data stream to trigger notifications intended to notify about deviations from expected workflow, process control or clinical course, as well as the ability to review and replay the sequence of data points in the past so that users can engage in a critical evaluation of a specific event or sequence of events that led to a negative clinical outcome, or non-compliance with or failure of a process control or workflow. The present invention has the ability to use historical data to generate guidances to manage clinical conditions or new process controls/workflows in real time and the ability for a user to acknowledge that a clinical guidance was true/valid in near real time.

The present invention has the ability to change execution pathway per user criteria depending on inputs in real time (e.g. data from a micro assessment could change the frequency of future assessments etc.), as well as enable end-users who are consuming the notifications of the improvement tool to direct and coordinate the team to change the input provided to the improvement tool at the time of the review of data so that the any updated workflow, for instance with additional evaluations, or modified evaluations.

The present invention has the ability to collect, store, and process data in near real-time. It can compose views that organize data for end-users to consume, to let the user create execution steps on the data streams, to notify end-users based on execution steps defined by end-users, and to present an organized view of data within a timeline of events. Further, the present invention has the ability for end-users to change or augment the execution steps and notifications at the same time the end-user is reviewing data.

The remote view system can include software means to review remotely all applicable data associated with the patient current condition, treatment and history to determine appropriate recommended patient care, means to define specific notification rules to define when to provide notification to any number of individuals required to be notified when the defined condition is met, means to allow persons to access to their personal schedule and/or the schedule of their subordinates, a dashboard to identify specific care areas, or patients of potential interest based on defined rules, means to review adherence to patient care plan and the patient's response to the treatment may be reviewed remotely with near real time data, and means to select any point in time associated with the patient record to assess patient response before and/or after that point in time.

The interface rules engine creates guidance rules one the end-user defines the criteria to be evaluated and the notifications that need to be delivered to the care team. End-user can define criteria based on any data-point available in the collected data stream, e.g., from medical devices. Users can be clustered into groups. Patients can be clustered into groups. Patients can be “tagged” with user defined criteria. Patients can be stratified according to user defined criteria in real time. Certain thresholds can be altered by an end user on the fly.

When and how communications are made regarding cases meeting defined criteria that need review occur when authorized End-users who use the “continuous improvement tool” have the ability to define the communication type (for instance, email notification to Cockpit user, sms, and who receives it. Also, when they receive it. Communication will be delivered on any device as prescribed by authorized end user in order to optimally support defined workflow. End-user also can configure the escalation process to execute if the notification is not acknowledged or addressed. End users will have ability to “snooze” certain communications if allowed by authorized end user.

Improvement is achieved every time end-users realize that there is a difference between the process followed by end-users and a better process that they could have followed based on best practice, peer-reviews publications or reviews of data collected and stored by the continuous improvement tool. But, this is complex. Errors and negative events will be identified dynamically. When this happens retrospectively, all data will be available (including waveforms) to enable the richest possible clinical review. New guidances can be created in real time if specific sentinel events or sequence of data are identified. Deviations form an expected process or workflow can be identified in real time. Following evaluation, any changes to a process control or workflow can be implemented dynamically

The remote device 12 can include any number of exemplar devices. Such exemplar devices include, but are not limited to, a mobile phone, a Smartphone, a personal digital assistant (PDA), a laptop, a tablet personal computer (PC), a desktop PC, and/or combinations thereof. The remote device 12 includes a display 22, a processor 24, memory 26, an input interface 28, and a communication interface 30. The processor 24 can process instructions for execution of implementations of the present disclosure. The instructions can include, but are not limited to, instructions stored in the memory 26 to display graphical information on the display 22. Exemplar displays include, but are not limited to, a thin-film-transistor (TFT) liquid crystal display (LCD), or an organic light emitting diode (OLED) display.

The memory 26 stores information within the remote device 12. In some implementations, the memory 26 can include a volatile memory unit or units, and/or a non-volatile memory unit or units. In other implementations, removable memory can be provided, and can include, but is not limited to, a memory card. Exemplar memory cards can include, but are not limited to, a secure digital (SD) memory card, a mini-SD memory card, a USB stick, and the like.

The input interface 28 can include, but is not limited to, a keyboard, a touchscreen, a mouse, a trackball, a microphone, a touchpad, and/or combinations thereof. In some implementations, an audio codec (not shown) can be provided, which receives audible input from a user or other source through a microphone, and converts the audible input to usable digital information. The audio codec can generate audible sound, Such as through a speaker that is provided with the remote device 12. Such sound may include, but is not limited to, Sound from Voice telephone calls, recorded Sound (e.g., voice messages, music files, etc.), and Sound generated by applications operating on the remote device 12. The remote device 12 may communicate wirelessly through the communication interface(s) 14, which can include digital signal processing circuitry. The communication interface(s) 14 may provide communications under various modes or protocols including, but not limited to, GSM voice calls, SMS, EMS or MMS messaging, CDMA, TDMA, PDC, WCDMA, CDMA2000, and/or GPRS. Such communication may occur, for example, through a radio-frequency transceiver (not shown). Further, the remote device can be capable of short-range communication using features including, but not limited to, Bluetooth and/or WiFi transceivers (not shown).

The remote device 12 communicates with the network 16 through the connectivity interface(s) 14. The connectivity interface(s) 14 can include, but is not limited to, a satellite receiver, cellular network, a Bluetooth system, a Wi-Fi system (e.g., 802.X), a cable modem, a DSL/dial-up interface, and/or a private branch exchange (PBX) system. Each of these connectivity interfaces 14 enables data to be transmitted to/from the network 16. The network 16 can be provided as a local area network (LAN), a wide area network (WAN), a wireless LAN (WLAN), a metropolitan area network (MAN), a personal area network (PAN), the Internet, and/or combinations thereof.

The present invention may be operational with numerous other general purpose or special purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in association with local and/or remote computer storage media including, by way of example only, memory storage devices.

The present invention allows a clinician to view permanently saved waveforms and physiologic data values from a central location. For example, if a waveform and physiologic data may need to be reviewed and signed by a clinician. Once the waveform and physiologic data has been signed, it is stored permanently in the historical queue.

The present invention allows for waveform data to viewed on any remote screen, including personal computers, tablets, smart phones, intelligent workstations, and the like. Upon request, the waveform of data related to the patient care record can be displayed, along with any additional quality data desired. The waveform replay would be available at any time after the data is stored. While displayed, any time point in the case being reviewed will be available.

For example, when selecting any time point in the case for review, the waveform data will be available along with other data that is part of the patient care record such as the following:

i. Pre-procedure evaluation
ii. Procedural steps taken thus far

### 1. Intubation

2. IV placement
3. Anesthesia started

### 4. Incision

### 5. Etc.

iii. Practitioner documented evaluations
iv. Medical device data including patient vital sign monitors.
b. Drug/fluid delivery details
i. Time (delivered or start, change and end)
ii. Quantity
iii. Changes in delivery, etc.
c. Supportive data from various data sources including
i. Labs
ii. Blood bank
iii. Patient medical history
iv. Pharmacy

The time selecting unit may include a cursor to select coordinate positions by allowing a user to move and position the cursor, and a cursor monitoring unit to acquire a time corresponding to the cursor's coordinate position while the cursor moves. The apparatus may further include a time display unit to display a time acquired by the cursor monitoring unit in the trend display graph.

The system further includes a data storage unit to store the measurement values of and waveforms of the physiological parameters

The following terms used in the description that follows. The definitions are provided for clarity of understanding:

Assessment data—assessment data is all data relevant to the health of a patient.

Healthcare location—A “healthcare location;” a facility, whether temporary or permanent, that is not generally equipped to provide expert medical care on a twenty-four basis. By way of illustration and not as a limitation, a healthcare location may be a remote clinic, a doctor's office, a field hospital, a disaster aid station, a patient transport vehicle and similar care facilities

Caregiver—an individual providing care to a patient. Examples include a nurse, a doctor, medical specialist (for example and without limitation an intensivist, cardiologist or other similar medical specialist).

Clinical data—data relating to the observed symptoms of a medical condition.

Monitored patient—a person admitted to a healthcare location.

Monitored data—data received from monitoring devices connected to a monitored patient. monitored patient—a monitored patient from whom monitored data is collected and whose condition is subject to continuous real-time assessment from a remote command center. patient data—data relating to a patient's diagnosis, prescriptions, history, condition, laboratory results and other health-relevant data.

Physiological data—any data relating to the functions of the human body and its processes. symptom—any sign or indication of a health condition that can be identified from patient reports and/or assessment data.

The present invention uses a telecommunications network to facilitate rules-based care of patients receiving care in a healthcare location. As used herein, a healthcare location may be a remote clinic, a doctor's office, a field hospital, a disaster aid station, a patient transport vehicle and similar care facilities. A patient may be selected for monitoring based on criteria established by the treatment facility. By way of illustration and not as a limitation, a “monitored patient” comprises a critically ill patient, an acutely ill patient, a patient with a specific illness, a patient with serious injuries, and a patient with an uncertain diagnosis.

Patient monitoring equipment acquires monitored data elements from a patient monitoring station and transmits the monitoring data over a network to a remote command center. Monitored data comprises physiological data elements, video data elements, and audio data elements. The remote command center receives the monitoring data from all patient monitoring stations. The remote command center also accesses other data relating to the condition of a patient. By way of illustration and not as limitation, the remote command center has access to data relating to personal information about the patient (name, address, marital status, age, gender, ethnicity, next of kin), medical history (illnesses, injuries, surgeries, allergies, medications), admissions information (symptoms, physiological data, time of admission, observations of admitting caregiver), treatment, lab data, test reports (radiology reports and microbiology reports for example), physician's notes, a patient's diagnosis, prescriptions, history, condition, laboratory results and other health-relevant data (collectively “patient data”) to the extent available from the healthcare location. The data available to the remote command center over the network, that is, the monitoring data and the patient data, is collectively referred to as “assessment data.”

In the present invention, a monitored patient care system provides care to monitored patients based on the capabilities of the healthcare location. The rules engine, the decision support algorithms, the order writing software facilities, and the continued care software are adapted to the capabilities of the healthcare location based on the application of site assessment rules to the healthcare location. In the present invention, components of a healthcare location patient care system may be supplied to the healthcare location to improve the level of its treatment capabilities.

Patient monitoring equipment acquires monitored data elements from a patient monitoring station and transmits the monitored data (sometimes also referred to herein as, “monitoring data”) over a network to a remote command center. Monitored data comprises physiological data elements, video data elements, and audio data elements. The remote command center receives the monitored data from all patient monitoring stations. The remote command center also accesses other data relating to the condition of a patient. By way of illustration and not as limitation, the remote command center has access to data relating to personal information about the patient (name, address, marital status, age, gender, ethnicity, next of kin), medical history (illnesses, injuries, surgeries, allergies, medications), admissions information (symptoms, physiological data, time of admission, observations of admitting caregiver), treatment, lab data, test reports (radiology reports and microbiology reports for example), physician's notes, a patient's diagnosis, prescriptions, history, condition, laboratory results and other health-relevant data (collectively “patient data”) to the extent available from the healthcare location. The data available to the remote command center over the network, that is, the monitored data and the patient data, is collectively referred to as “assessment data.”

A rules engine applies a rule or rule set to the data elements selected from the assessment data from each monitored patient to determine whether the rule for that site has been contravened. In the event the rule has been contravened, an alert at the remote command center is triggered. Rules for each monitored patient may be established and changed at the remote command center for each as the patients' conditions warrant. In one embodiment of the present invention, a rule is established to determine whether a patient's condition is deteriorating. An alert that a rule has been contravened comprises advice on treatment of the patient.

A patient rules generator establishes one or more rules for the monitored patient associated with a patient monitoring station. The patient rules generator collects rules performance measures indicative of the ability of the rule to predict changes in the condition of a patient and uses these measures to assess the efficacy of the rule. The patient rules generator may update a rule, determine that a rule is acceptable as is, or determine that there is insufficient data to revise a rule.

The patient rules generator may also evaluate the assessment data of patients with similar conditions to determine whether a predictive rule can be written and applied to patients with the same or similar conditions. The patient rules generator may also test a proposed rule against historical data to determine whether the rule is predictive of a change in a patient's condition. The patient rules generator generates a rule that is consistent with the service level measures established by a site assessment module.

The present invention provides continued care software that uses elements of the assessment data to provide decision support and that prompts a user for input to provide decision support to caregivers. A decision support algorithm responds to elements of assessment data to produce textural material describing a medical condition, scientific treatments and possible complications. This information is available in real time to assist in all types of clinical decisions from diagnosis to treatment to triage.

In the present invention, a healthcare location patient care system provides care to healthcare location patients based on the capabilities of the healthcare location. In this embodiment, the rules engine, the decision support algorithms, the order writing software facilities, and the continued care software are adapted to the capabilities of the healthcare location based on the application of site assessment rules to the healthcare location. Components of a healthcare location patient care system may be supplied to the healthcare location to improve the level of its treatment capabilities. In still another embodiment of the present invention, components of the healthcare location are packaged and assigned a site assessment code. The code is used by the remote command center to predetermine elements of the site assessment process thereby simplifying that process.

In the present invention, patient-monitoring equipment acquires monitored data elements from a patient monitoring station and stores monitoring data locally. The stored monitoring data is sent to a remote command center along with patient data at a pre-established time or when requested by remote command center. The remote command center evaluates the “delay” monitored data and assessment data in the same manner as if these data were received in real time. By way of illustration, the remote command center will apply the rules engine and the decision support algorithms to the delayed monitored data and patient data and provide guidance to the healthcare location. The present invention thus provides high quality care in environments where continuous high bandwidth communications are not available or economically infeasible.

The system of the present invention collects the data and stores the data. The patient record in the medical facility electronic medical record system (EMR) contains the same information as is known in the art with other systems. The EMR is one target and receives only the data required by the specific target. The data sent to the EMR plus any other data collected is stored in the cloud and filed support the assessment of the case. There is the ability to store some of the data as case record and other additional data collected in the Quality data. The potential claims being addressed here is another example of how we continue to add functionality and features by finding new ways to more fully leverage the knowledge we are gaining as we gain knowledge. This is a perfect example of how the system has the ability to grow as new knowledge is gained.

The invention is capable of collecting any identified data at a frequency that supports a meaningful assessment of patient interactions and patient response to those interactions. The term data includes but is not limited to patient medical history, patient interaction details including person performing the interaction, the time provided, any drug, disposable or medical device used to complete the interaction, numeric patient vital signs provided by active patient monitors, Waveforms provided by active patient monitors, changes to settings of any medical device used on the patient, lab results, practitioner notes and documented observations.

The invention is capable of displaying the information along a time line of the treatment period. The display may be configured to provide a graphical representation of any numeric patient vital sign collected during the treatment. Additionally, the invention supports the viewing of the waveform data generated during the treatment as an accurate representation of the waveform screens on the active patient monitors.

Using the review screen the user may select any specific time on the time line to review the patient vital signs values at that time. With respect to numeric and waveform data the system has the ability to move forward and backward during the treatment to assess potential link between patient interaction and patient response.

Using these tools, the person or team conducting the assessment may identify potential root causes and or potential corrective/preventive actions including the establishment of guidances. The system is designed to allow the user to establish parameter or conditional limits to implement guidances. In this case implement implies that the system will monitor future treatments (same treatment as reviewed) against specified conditions and when any of those conditions are observed, an alert/notification will be sent. The level of implementation may be limited to monitoring in the back ground without any interaction with the provider. In this case the individual(s) identified by the guidance will be notified that a treatment case of interest is available for review. Note: It is possible for several guidances to be created and implemented to accelerate the collection of data for various alternative approaches. Using this approach, the data necessary to build the required scientific evidence to justify the appropriate treatment protocol guidance may be collected without any change to currently approved protocols.

Once the scientific evidence has been collected to justify a formal change to the Medical Facility's protocol, the appropriate review can be performed and if adequately justified, approved for implantation via changes to the authorized protocol. As knowledge is gained using this basic design of experiment methodology, sufficient logic may be developed to support the use of machine learning tools to further accelerate the continuous improvement program.

The present invention supports the ability to move back in time during the current patient care under review. The same screens may also be utilized to evaluate the case during formal reviews. These screens also include icons to show when specific patient interactions were performed.

The waveform data is collected directly from the medical devices. These waveforms are stored with the patient records. Once stored the waveforms may be displayed and played. With the ability to play the stored waveforms the system is also includes to play, replay, fast forward or fast reverse to identify the time periods of most interest to the medical practitioner reviewing these records. Continuous improvement program focused on patient outcome: Improved patient outcome requires a continuous spiral of improved process control. Therefore, continuous improvement programs focus of identifying opportunities to eliminate unexpected outcomes.

The present invention looks for situations where the existing process controls or procedures yield negative outcomes. Truly advanced programs also look for situations where the outcomes are more positive than expected. When situations presenting opportunities for improvement are identified, the organization determines the risk of a repeat event and prioritizes resources to address the top opportunities. The identified situations are tracked and managed in the Corrective and Preventive Action process. This process requires documented Failure Investigation details, assessment of potential corrective and/or preventive actions, verification and validation outcomes of the C/P actions tried. As discussed above, the current prior art systems do not adequately support an effective Continuous Improvement Program.

### Example of the Remote View Tool:

Remote view supports telemedicine by providing the remote practitioner with near real time access the patient's record and ability to review all vital signs, patient care activities and patient response to the treatment leading up to the current condition. This provides for a more complete understanding of the patient condition. An example of this is as Anesthesiologist supervising multiple CRNA's. Remote view provides the supervisor to review the facts regarding a specific patient while walking to the OR to assist the CRNA. Thus, reducing the time required to review the patient status with the CRNA prior to taking the appropriate actions to address the situation.

The system of the present invention will best be used when there is a specific reason to analyze the details of the patient's condition remotely. Examples include: providing physicians with the ability to: check up on their patients remotely, improve support of attending practitioners under their supervision, prioritize their schedules based on the current condition of their patients, and provide remote telemedicine intensivists the ability to review events leading up to the current patient condition.

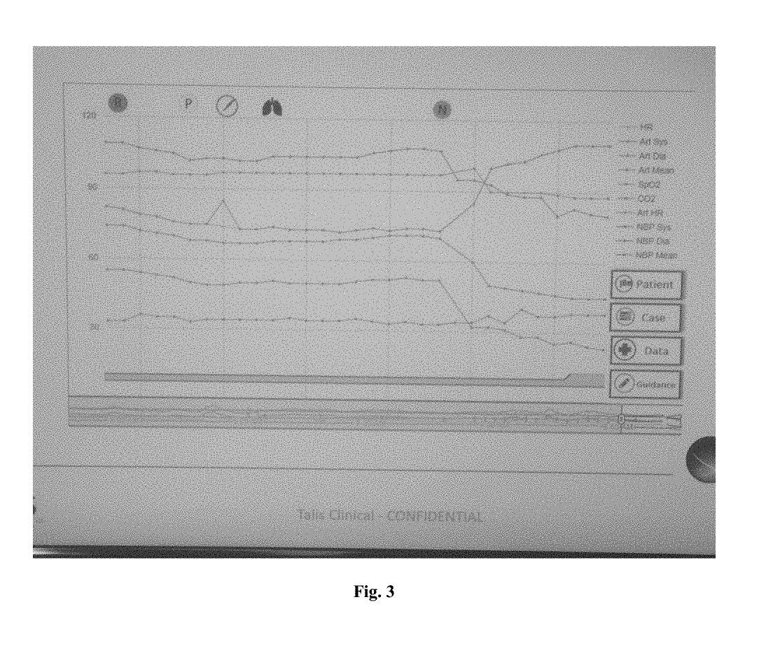

FIGS. 3-12 provide an example for an anesthesia protocol. However, the example and its slides and explanation of each screen are not intended to limit the scope of the capabilities of the Continuous Improvement Tool. The tool may be configured to support any number of processes.

In the present example of a case analysis, a review screen is provided that provides on overview of the case including, but not limited to:
Graphical representation of the patient vital signs during the case, including icons indicating when specific interaction with patient occur
   Legend of the graphs
   Drug delivery
   Incision
   Ventilation
   Case Detail Selection Keys
   Patient—details
   Case
   Data
   Guidance

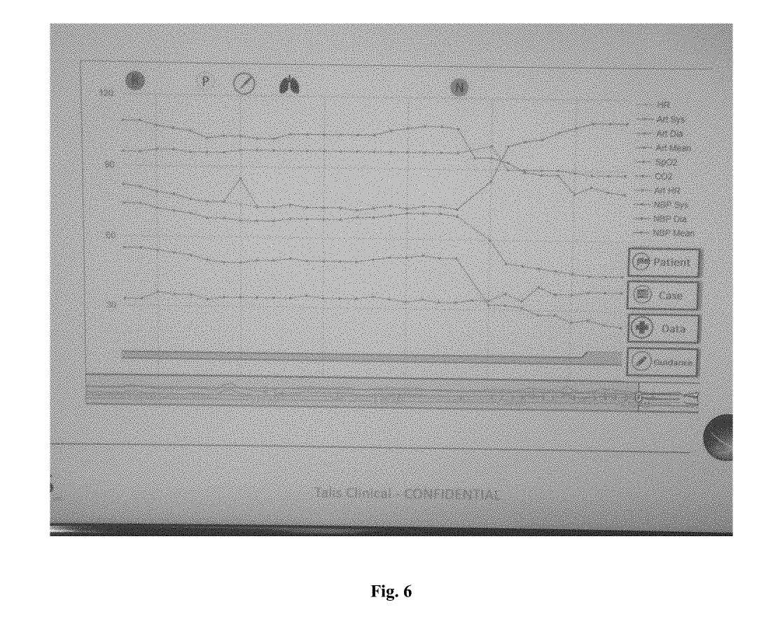

The screenshots provided walk through an example where data collected through a case is reviewed. In the timeline, data from multiple sources is visible along with clinical events, (i.e. incision) The screenshots FIGS. 3,4, and 5 show how clinicians interactively review data along with the guidance editor. Then can evaluate their current process controls (FIG. 6) and make changes to processes based on the review of the case. The screenshots show an example of how this happens. For example, during a review clinicians might identify that the data showed in the screens of the review tool might be more effective if it also showed data from available, from cerebral oximetry. Because the platform supports Cerebral monitors, clinicians can request to include cerebral oxymeters in the data set collected, so that clinicians can review and use its data to create more effective guidance. The remote view is not limited to the EMR data, the EMR contains a subset of the data available for remote view analysis.

FIG. 3 is a screen shot of a review screen showing an interaction with the review screen data options, including an ability to move the cursor to a specific time in the case and clicks to see specific data details. This screen shot displays the actual collected data at the requested time of the case.

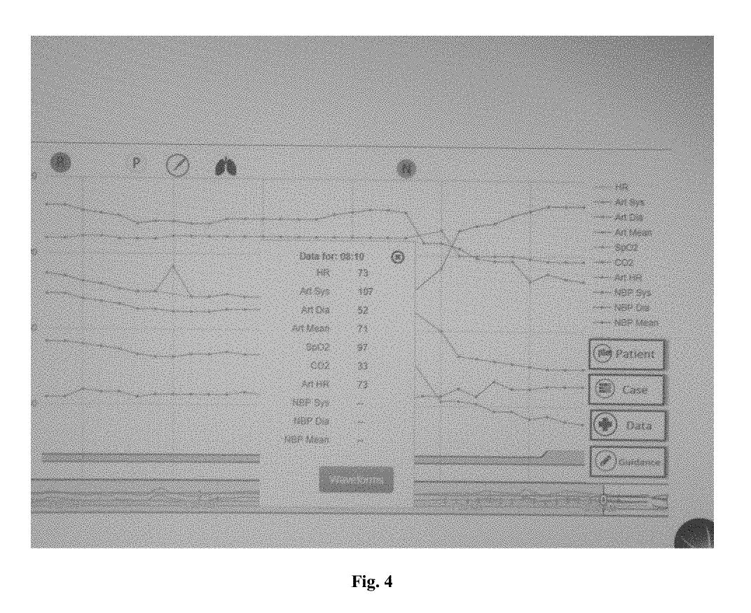

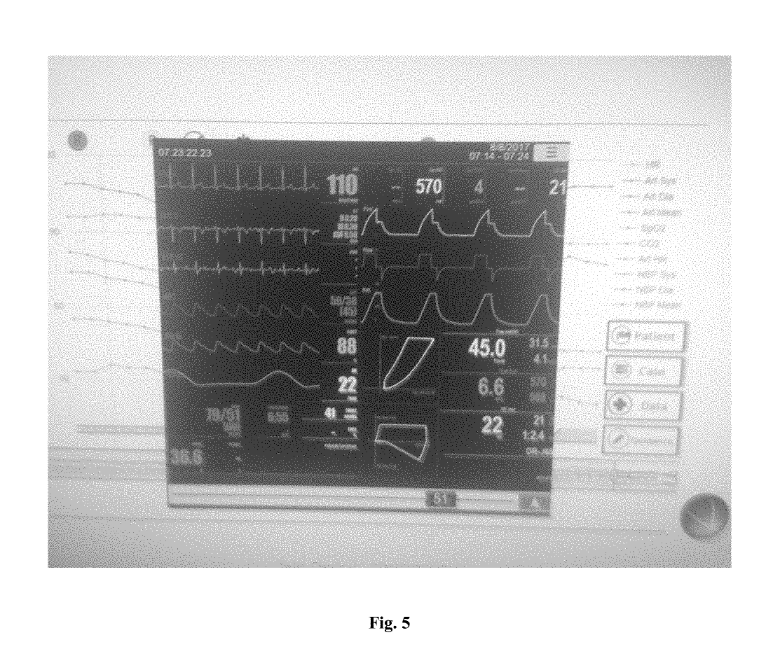

To see waveforms the user clicks on the waveform key, as shown in the screen shot in FIG. 4 and the waveform data collected is displayed as shown in the screen shot in FIG. 5.

As seen in the screen shot is FIG. 6, there is a slide at the bottom of the waveform display, which enables the user to move the time back or forward to review changes before or after the selected time.

The screen shots illustrate how, from the review screen the user can access the guidance tool which walks the user through the process to create or modify a guidance.

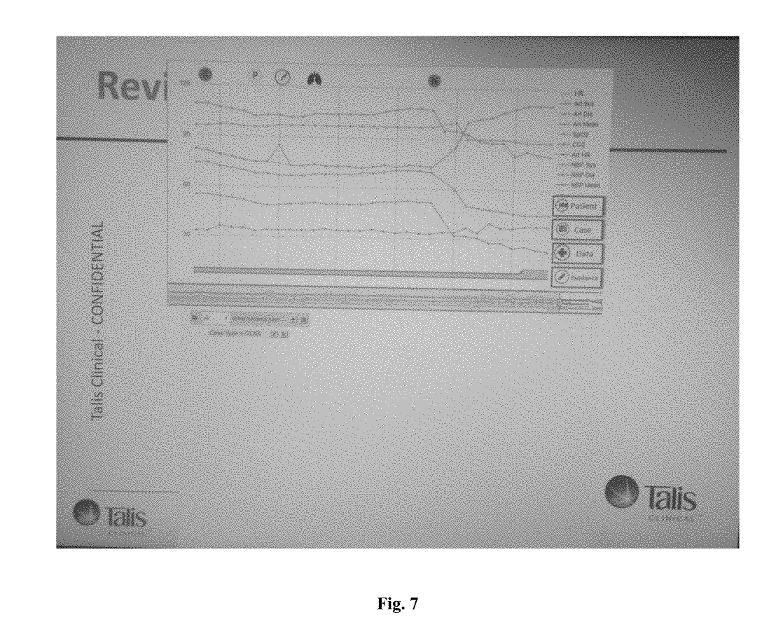

FIG. 7 shows the review screen and the selection of the scope of the primary filter which in this case defines the case type a GENA.

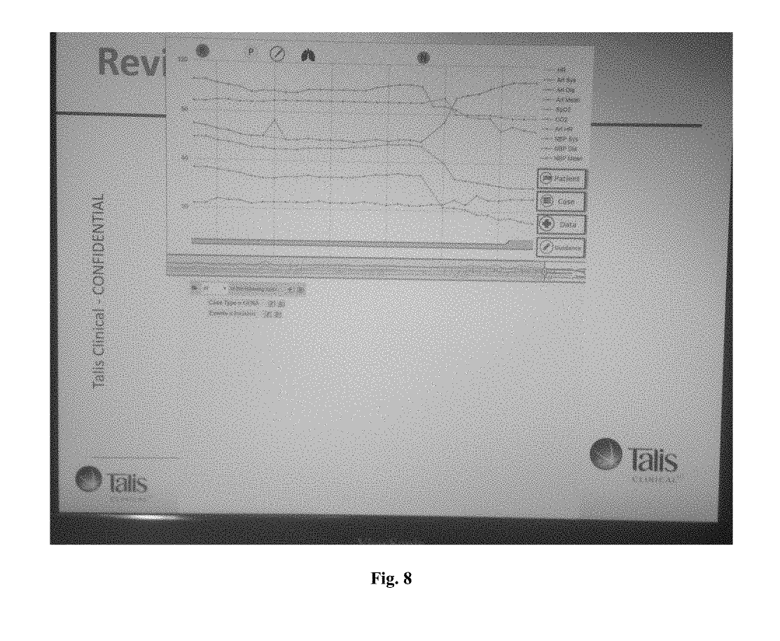

FIG. 8 shows the review screen and patient interactions, as well as showing that the event is an incision.

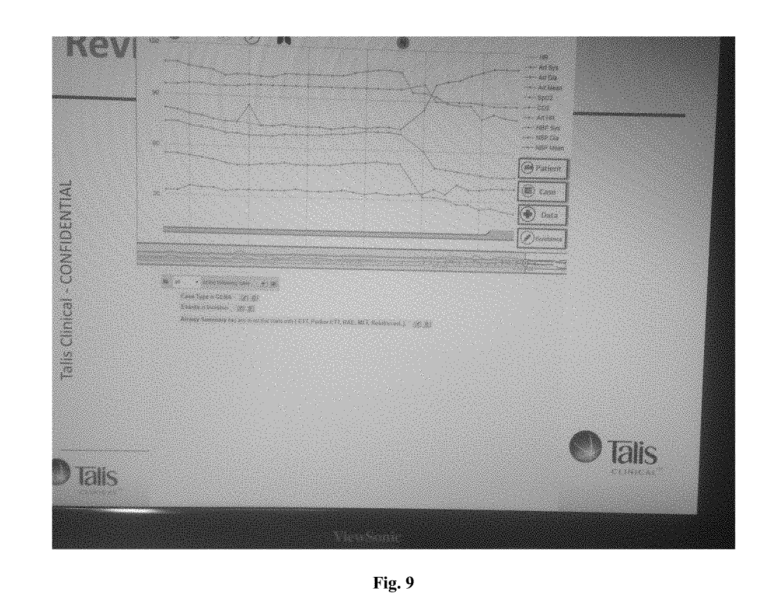

FIG. 9 shows a screen shot of an airway summary and has any list that starts with ETT, Parker ETT, RAE, and MLT and/or reinforced.

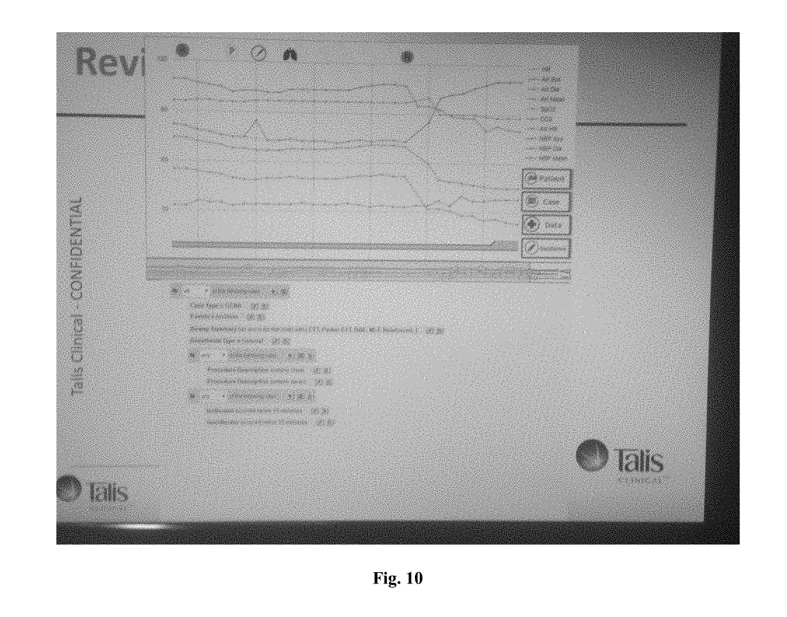

FIG. 10 shows a screen shot indicating that the anesthesia type is general and defines the scope of secondary filter according to certain rules, including whether the procedure description contains crani and/or the procedure description contains neuro. FIG. 10 also shows the definition of the 3rd level filter, and indicates that Isoflurane occurred before 15 minutes and Sevoflurane occurred before 15 minutes.

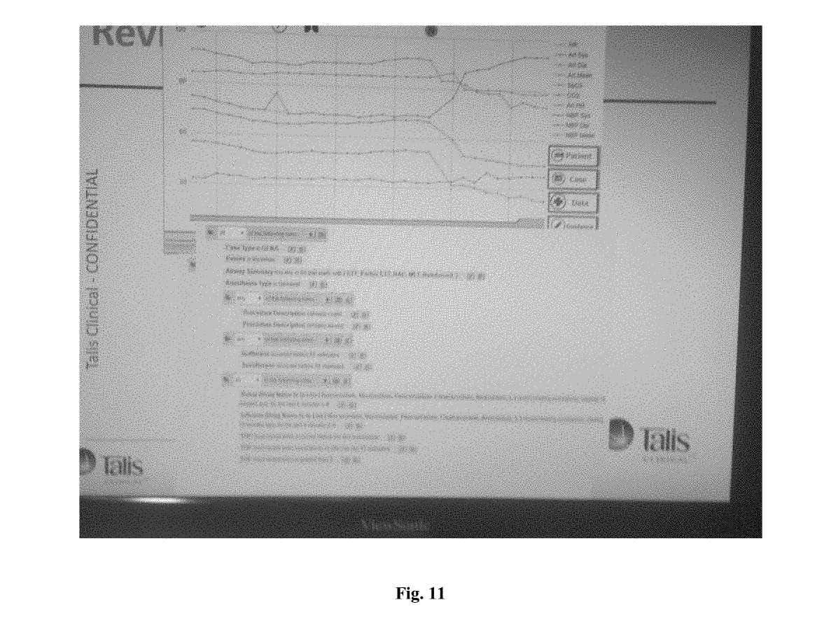

FIG. 11 shows the scope of a 4th level filter as defined by the following rules;
Bolus (Drug Name is in List (Rocuronium, Vecuronium, Pancuronium, Atracurium),) recent reading occurrences starting 10 minutes ago for the last 9 minutes is 0
Infusion (Drug Name is in List (Rocuronium, Vecuronium, Pancuronium, Atracurium),) recent reading occurrences starting 10 minutes ago for the last 9 minutes is 0
TOF most recent entry occurred before the last 5 minutes TOF most recent entry occurred on or after the last 11 minutes
TOF most recent entry is greater than 2.

The foregoing embodiments of the present invention have been presented for the purposes of illustration and description. These descriptions and embodiments are not intended to be exhaustive or to limit the invention to the precise form disclosed, and obviously many modifications and variations are possible in light of the above disclosure. The embodiments were chosen and described in order to best explain the principle of the invention and its practical applications to thereby enable others skilled in the art to best utilize the invention in its various embodiments and with various modifications as are suited to the particular use contemplated.

What we claim is:

1. A remote view playback system for medical patients to improve patient outcomes comprising:
a telecommunications network;
at least one monitoring station comprising monitoring equipment wherein the monitoring equipment includes instructions for monitoring data elements and for sending the monitored data elements via the telecommunications network, and includes instructions for receiving monitored data elements from patients and accessing patient data elements indicative of a medical conditions associated with each of the patients;
a patient database containing information concerning the medical condition, history, and status of each of the patients;
at least one communication hub comprising instructions for collecting data from any number of electronic devices including medical devices and instructions for transmitting data to any number of electronic devices including medical devices, as well as instructions for storing data associated with the patient records and/or data to be stored as quality data;
a data storage engine comprising a means for collecting data from any number of electronic devices including medical devices and instructions for transmitting data to any number of electronic devices including medical devices, as well as instructions for storing data associated with the patient records and/or data to be stored as quality data;
said telecommunication network providing access to all data, including continuous waveform data, collected during the treatment of the patient and quality data not included in any patient record storage location; and
a user interface rules engine that provides the user with ability to select any point in time during the patient treatment to:
i. review the details collected regarding the treatment at the selected time
ii. review the details before or after the selected time.
iii. create guidance rules to identify cases identified as complying with the defined rules, the user may define the period of time used to identify the cases for review, the user may select future cases only, or past cases to some defined date, or a combination of the two.
iv. define who, when and how to communicate that cases meeting the defined criteria are available for review, in this case the who may be only the individual(s) evaluating the defined criteria without notification to anyone monitoring a current case, or including specific individuals monitoring the current case;
wherein said user interface rules engine includes:
means to collect, store and process data in near real-time,
means to compose views that organize data for end-users to consume,
means to let a user create execution steps on the data streams,
means to notify end-users based on execution steps defined by end-users,
means to display an organized view of data within a timeline of events,
means for end-users to change or augment the execution steps, and
means to provide notifications at the same time the end-user is reviewing data.
whereby data from multiple disparate sources can be acquired and consolidated within a unified view, and the data can be reviewed remotely to make decisions about patient care.

2. The system of claim 1 wherein the system identifies which information is actionable using an algorithm based upon user criteria.

3. The system of claim 1 further including means to review remotely all applicable data associated with the patient current condition, treatment and history to determine appropriate recommended patient care.

4. The system of claim 1 further including means to define specific notification rules to define when to provide notification to any number of individuals required to be notified when the defined condition is met.

5. The system of claim 1 further including means for selecting any point in time associated with the patient record to assess patient response before and/or after that point in time

6. The system of claim 1 further including means for reviewing remotely, adherence to patient care plan and patient's response to the treatment, with near real time data.

7. The system of claim further including a time display unit to display a time acquired by the cursor monitoring unit in the trend display graph.

8. The system of claim 1 further including means to access to a personal schedule and/or the schedule of their subordinates.

9. The system of claim 1 further including a dashboard to identify specific care areas, or patients of potential interest based on defined rules.

10. A method of remotely viewing records of medical patients to improve patient outcomes comprising:
providing a telecommunications network;
providing at least one monitoring station comprising monitoring equipment wherein the monitoring equipment includes instructions for monitoring data elements and for sending the monitored data elements via the telecommunications network, and includes instructions for receiving monitored data elements from patients and accessing patient data elements indicative of a medical conditions associated with each of the patients;
providing a patient database containing information concerning the medical condition, history, and status of each of the patients;
providing at least one communication hub comprising instructions for collecting data from any number of electronic devices including medical devices and instructions for transmitting data to any number of electronic devices including medical devices, as well as instructions for storing data associated with the patient records and/or data to be stored as quality data;
providing a data storage engine comprising a means for collecting data from any number of electronic devices including medical devices and instructions for transmitting data to any number of electronic devices including medical devices, as well as instructions for storing data associated with the patient records and/or data to be stored as quality data;
said telecommunication network providing access to all data, including continuous waveform data, collected during the treatment of the patient and quality data not included in any patient record storage location; and
providing a user interface rules engine that provides the user with ability to select any point in time during the patient treatment for:
i. reviewing the details collected regarding the treatment at the selected time
ii. reviewing the details before or after the selected time.
iii. creating guidance rules to identify cases identified as complying with the defined rules, the user may define the period of time used to identify the cases for review, the user may select future cases only, or past cases to some defined date, or a combination of the two.
iv. defining who, when and how to communicate that cases meeting the defined criteria are available for review, in this case the who may be only the individual(s) evaluating the defined criteria without notification to anyone monitoring a current case, or including specific individuals monitoring the current case;
wherein said user interface rules engine includes:
means to collect, store and process data in near real-time,
means to compose views that organize data for end-users to consume,
means to let a user create execution steps on the data streams,
means to notify end-users based on execution steps defined by end-users,
means to display an organized view of data within a timeline of events,
means for end-users to change or augment the execution steps, and
means to provide notifications at the same time the end-user is reviewing data,
whereby data from multiple disparate sources can be acquired, consolidated within a unified view, process controls and workflows can be run, and actionable insights can be delivered to specific users in near-real time.

11. The method of claim 10 wherein the method further includes an apparatus for waveform analysis of physiological parameters, the apparatus including a physiological parameter acquisition unit and a display device, and the method includes the steps of:
reading, using the physiological parameter acquisition unit, measurement values and corresponding times of physiological parameters, and displaying a plurality of variations in the measurement values of the physiological parameters over time as a trend display graph in a trend display area of the display device, the trend display area including coordinates that represent the times and the measurement values, respectively, wherein displaying the plurality of variations in the measurement values with respect to the second coordinate in the trend display area indicates a trend in the first physiological parameter throughout a monitoring period;
storing the measurement values of the physiological parameters, the waveform data of the physiological parameters, pathologic event data, and a time corresponding to when a pathologic event takes place, in a data storage device; and
setting the time corresponding to when the pathologic event takes place as a primary key to build an index logic table including data and time so as to associate the data during the storing step.
